(19) United States
(12) Patent Application Publication
(10) Pub. No.: US 2025/0053588 A1
(43) Pub. Date: Feb. 13, 2025

(54) ARTIFICIAL INTELLIGENCE BASED (AI-BASED) COMPUTING SYSTEM AND METHOD FOR ACCREDITATION OF INDUSTRIAL PROFESSIONALS

(71) Applicant: Rajeshwari Kartik, Allen, TX (US)

(72) Inventor: Rajeshwari Kartik, Allen, TX (US)

(21) Appl. No.: 18/926,448

(22) Filed: Oct. 25, 2024

Related U.S. Application Data
(63) Continuation-in-part of application No. 17/476,629, filed on Sep. 16, 2021, now abandoned.

(51) Int. Cl. G06F 16/335; G06F 16/33

(52) U.S. Cl. CPC G06F 16/337; G06F 16/3334

(57) ABSTRACT

An AI-based computing system and method for accreditation of industrial professionals is disclosed. The AI-based computing system includes plurality of subsystems that includes a profile generation subsystem configured to create user profiles for registered users using user credentials. The plurality of subsystems includes an event manager subsystem configured to (a) provide access to continued industrial education content to registered users based on created user profiles, (b) provide AI-based personalized recommendations on the continuing industrial education content in priority using AI model, and (c) manage tasks associated with user profile relating to continuing industrial education content responsive to providing the access. The plurality of subsystems includes a credit generation subsystem configured to generate credit scores for the user profiles based on the tasks completed by registered users, using the AI model. The plurality of subsystems includes a report generation subsystem configurate to generate and publish a report on tasks.

### CROSS-REFERENCE TO RELATED APPLICATIONS

The present application is a continuation in part of U.S. patent application Ser. No. 17/476,629, filed Sep. 16, 2021, titled “SYSTEM AND METHOD FOR ACCREDITATION OF INDUSTRIAL PROFESSIONALS”.

### FIELD OF INVENTION

Embodiments of the present disclosure relates to the technical field of artificial intelligence (AI-based) information management systems, and more particularly to an artificial intelligence based (AI-based) computing system and a method for accreditation of industrial professionals.

### BACKGROUND

Today every industrial field is developing at a rapid pace and the associated depth of knowledge is increasing every moment. Therefore, it becomes essential for an industrial professional to continuously become acquainted with the latest developments in their respective industrial field. The industrial field can include medical, engineering, legal, and the like. The rapid pace of industrial advances makes it mandatory for professionals to keep themselves updated such that they may apply this information to their practice and thus fortify their competence and knowledge by staying updated with latest developments in their field.

The industrial professionals need to maintain their knowledge, skills, competence and performance over time and hence, they need to continue their industrial education. Many jurisdictions require continuing industrial education for each industrial professional to maintain their industrial license. For example, continuing medical education (CME) is one such type of Continuing Industrial Education. Such education methods are based on credit points or hours. Various educational councils have accepted such methods of learning to provide licences and accreditation.

Conventionally, there is a lack of a common platform for industrial professionals and industrial organizations and institutes in accessing information related to continuing industrial education and accreditation related that the same. Moreover, systems currently available lack the integration of varied data sources for access by industrial professionals. Real time calculation mechanism for the credit points or hours are also desirable for distributing industrial licenses.

Hence, there is a need for an improved system for the accreditation of industrial professionals and a method to operate the same and therefore address the aforementioned issues.

### SUMMARY

In accordance with one embodiment of the disclosure, an artificial intelligence based (AI-based) computing system for accreditation of industrial professionals, is disclosed. The system comprising a hardware processor. The system a memory coupled to the hardware processor. The memory comprises a set of program instructions in the form of a plurality of subsystems. The plurality of subsystems is configured to be executed by the hardware processor.

The plurality of subsystems includes a profile generation subsystem configured to create a user profile for each of one or more registered users using one or more user credentials. The one or more registered users belong to one or more industries. The profile generation subsystem is further configured to assign an identification label to each of the created user profile. The identification label comprises contact details of the one or more registered users and an industrial license number. The identification label is associated with at least one of: a bar code, a quick response (QR) code, a numeric code, an alpha-numeric code, and a graphical code.

The profile generation subsystem is further configured to provide access to continuing industrial education content to the one or more registered users based on the created user profile. The computer-implemented system comprises an information crawler configured to scan, identify, and collect information related to the continuing industrial education content and associated activities occurring in a specified location. In an embodiment, providing access comprises a keyword searching module and a filtering module. The keyword searching module and the filtering module are augmented to generate optimized search recommendations based on at least one of: the created user profile and one or more behavioral patterns of the one or more registered users, using a natural language processing model.

The profile generation subsystem is further configured to provide AI-based personalized recommendations on the continuing industrial education content in priority using an AI model trained to predict user preferences based on one or more historical data comprising at least one of: one or more industry types, one or more user interests, one or more geographic locations, and one or more educational goals. The profile generation subsystem is further configured to manage one or more tasks associated with the user profile relating to the continuing industrial education content responsive to providing the access. In an embodiment, managing the one or more tasks comprises a continuous development reckoner. The continuous development reckoner achieves set targets, provides license renewal support, and plans specific number of knowledge and training programs.

The plurality of subsystems further includes a credit generation subsystem configured to generate a credit score for each of the user profile based on the one or more tasks completed by the one or more registered users, using the AI model.

The plurality of subsystems further includes a professional development score generation subsystem configured to generate professional development score for each of the user profile based on one or more factors upon completion of the one or more tasks using the AI model. The one or more factors comprises at least one of: continuing industrial education history, number of years of schooling and college education, total levels of continuing professional development courses, and employment history.

The plurality of subsystems further includes a report generation subsystem configured to (a) generate one or more reports on each of the one or more tasks completed by the one or more registered users relating to the continuing industrial education content and (b) publish the generated one or more reports to the one or more registered users and other industrial authorities via a communications network.

In an embodiment, the artificial intelligence based (AI-based) computing system further comprises a training subsystem configured to train the AI model for providing the AI-based personalized recommendations on the continuing industrial education content in priority. For training the AI model, the training subsystem is configured to: (a) obtain one or more training datasets associated with the one or more historical data from one or more databases; (b) train the AI model on the one or more training datasets associated with the one or more historical data; (c) generate one or more scores for relevancy of each continuing industrial education content based on the trained AI model; (d) assign one or more weightages to each continuing industrial education content based on the one or more scores generated for each continuing industrial education content; (e) provide the AI-based personalized recommendations on the continuing industrial education content, in priority, to predict the user preferences, based on the one or more weightages assigned to each continuing industrial education content; and (f) refine the AI model on the prediction of the user preferences through a feedback mechanism based on user activities with the AI-based personalized recommendations on the continuing industrial education content.

In another embodiment, the one or more tasks of the user profiles comprises listing conferences on which each of the one or more registered users is interested in, a time, a date and a place for each conference, and developing, delivering and organizing events by an industrial representative of an associated organization.

In yet another embodiment, the credit score is generated based on a number of meetings, conferences and courses accessed by each of the one or more registered users.

In yet another embodiment, for generating the credit score for each of the user profile based on the one or more tasks, the credit generation subsystem is configured to analyze the completed one or more tasks using one or more pre-defined weights assigned to each task based on one or more parameters comprising at least one of: complexity of the one or more tasks, relevancy of the one or more tasks to one or more industries, the one or more registered users belong to, time spent on completion of the one or more tasks. In an embodiment, the AI model is learned with the one or more pre-defined weights from one or more historical performance data associated with at least one of: the one or more registered users and one or more industries.

In yet another embodiment, the artificial intelligence based (AI-based) computing system further comprises a database configured to store information related to each of the one or more registered users and information related to the continuing industrial education content.

In yet another embodiment, the continuing industrial education content comprises continuing medical education.

In yet another embodiment, the training subsystem is further configured to train the AI model for generating the professional development score for each of the user profile. For training the AI model, the training subsystem is configured to: (a) obtain one or more second training datasets associated with at least one of: one or more professional development trajectories and one or more accrediting factors, from the one or more databases; (b) train the AI model on the one or more second training datasets associated with at least one of: the one or more professional development trajectories and the one or more accrediting factors; (c) generate one or more scores for the one or more factors comprising at least one of: the continuing industrial education history, the number of years of schooling and college education, the total levels of the continuing professional development courses, and the employment history, based on the trained AI model on the one or more second training datasets; (d) assign one or more second weightages for the one or more factors based on the one or more scores generated for the one or more factors; (e) generate the professional development score for each of the user profile based on the one or more second weightages assigned for the one or more factors; and (f) adapt the AI model to learn and enhance a process of generating the one or more scores for the one or more factors by adding one or more data comprising one or more user feedback and industry-specific standards.

In yet another embodiment, the continuing industrial education content comprises knowledge regarding continuing industrial education content, spreading awareness regarding a continuing industrial education program, activities of live events, written publications, online programs, audio, video, and other electronic media and activities comprising developing, reviewing, and delivering content regarding continuing industrial education.

In yet another embodiment, the event manager subsystem is further configured for prompting the one or more registered users to renew an industrial license with an industrial organization based on a time of expiration of the industrial licenses.

In accordance with one embodiment of the disclosure, an artificial intelligence based (AI-based) computing method for accreditation of industrial professionals, is disclosed. The AI-based computing method includes creating, by one or more hardware processors, a user profile for each of one or more registered users using one or more user credentials, wherein the one or more registered users belong to one or more industries. The AI-based computing method further includes assigning, by the one or more hardware processors, an identification label to each of the created user profile. In an embodiment, the identification label comprises contact details of the one or more registered users and an industrial license number, and wherein the identification label is associated with at least one of: a bar code, a quick response (QR) code, a numeric code, an alpha-numeric code, and a graphical code.

The AI-based computing method further includes providing, by the one or more hardware processors, access to continuing industrial education content to the one or more registered users based on the created user profile. The AI-based computing method further includes scanning, identifying and collecting, by the one or more hardware processors, information related to the continuing industrial education content and associated activities occurring in a specified location based on an information crawler. The AI-based computing method further includes augmenting, by the one or more hardware processors, a keyword searching module and a filtering module, to generate optimized search recommendations based on at least one of: the created user profile and one or more behavioral patterns of the one or more registered users, using a natural language processing model.

The AI-based computing method further includes providing, by the one or more hardware processors, AI-based personalized recommendations on the continuing industrial education content in priority using an AI model trained to predict user preferences based on one or more historical data comprising at least one of: one or more industry types, one or more user interests, one or more geographic locations, and one or more educational goals. The AI-based computing method further includes managing, by the one or more hardware processors, one or more tasks associated with the user profile relating to the continuing industrial education content responsive to providing the access. In an embodiment, managing the one or more tasks comprises a continuous development reckoner. The continuous development reckoner achieves set targets, provides license renewal support, and plans specific number of knowledge and training programs.

The AI-based computing method further includes generating, by the one or more hardware processors, a credit score for each of the user profile based on the one or more tasks completed by the one or more registered users, using the AI model. The AI-based computing method further includes generating, by the one or more hardware processors, professional development score for each of the user profile based on one or more factors upon completion of the one or more tasks using the AI model. The one or more factors comprises at least one of: continuing industrial education history, number of years of schooling and college education, total levels of continuing professional development courses, and employment history.

The AI-based computing method further includes generating, by the one or more hardware processors, one or more reports on each of the one or more tasks completed by the one or more registered users relating to the continuing industrial education content. The AI-based computing method further includes publishing, by the one or more hardware processors, the generated one or more reports to the one or more registered users and other industrial authorities via a communications network.

In another aspect, a non-transitory computer-readable storage medium having instructions stored therein that, when executed by a hardware processor, causes the processor to perform method steps as described above.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram illustrating an exemplary artificial intelligence based (AI-based) computing system for accreditation of industrial professionals, in accordance with an embodiment of the present disclosure;

FIG. 2 is a block diagram illustrating an exemplary computing environment comprising a user device and a server device communicating through a communication network, in accordance with an embodiment of the present disclosure;

FIG. 3 is a block diagram illustrating another exemplary AI-based computing system for accreditation of industrial professionals, in accordance with an embodiment of the present disclosure;

FIG. 4 illustrates a block diagram of components of the AI-based computing system and the interaction among themselves, in accordance with an embodiment of the present invention;

FIG. 5 is a flowchart illustrating an exemplary process of computing the professional development score, in accordance with an embodiment of the present disclosure;

FIG. 6 is a block diagram illustrating components in the AI-based computing system, such as those shown in FIG. 1, in accordance with an embodiment of the present disclosure; and

FIG. 7 is a process flowchart illustrating an AI-based computing method for accreditation of industrial professionals, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated online platform, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or subsystems or elements or structures or components preceded by “comprises . . . a” does not, without more constraints, preclude the existence of other devices, subsystems, elements, structures, components, additional devices, additional subsystems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

A computer system (standalone, client or server computer system) configured by an application may constitute a “subsystem” that is configured and operated to perform certain operations. In one embodiment, the “subsystem” may be implemented mechanically or electronically, so a subsystem may comprise dedicated circuitry or logic that is permanently configured (within a special-purpose processor) to perform certain operations. In another embodiment, a “subsystem” may also comprise programmable logic or circuitry (as encompassed within a general-purpose processor or other programmable processor) that is temporarily configured by software to perform certain operations.

Accordingly, the term “subsystem” should be understood to encompass a tangible entity, be that an entity that is physically constructed permanently configured (hardwired) or temporarily configured (programmed) to operate in a certain manner and/or to perform certain operations described herein.

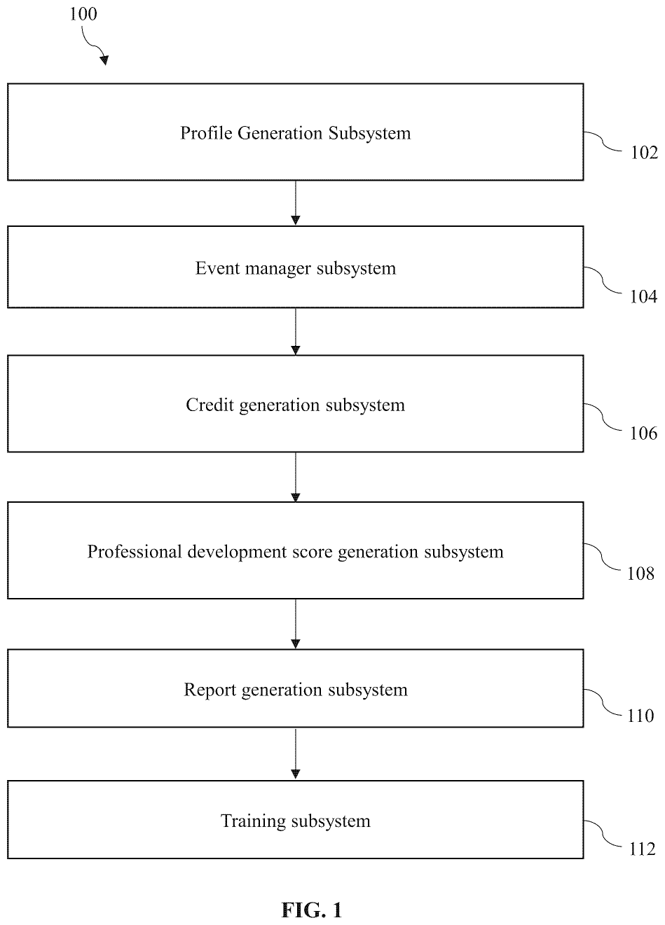

FIG. 1 is a block diagram illustrating an artificial intelligence based (AI-based) computing system 100 (hereinafter referred as a system) for accreditation of industrial professionals, in accordance with an embodiment of the present disclosure. The system 100 provides a common platform to the industrial professionals and industrial organizations, such as hospitals, institutions, legal institutes, engineering institutes and the like to share and access information related to continuing industrial education, related activities occurring, and accreditation associated with attending continuing industrial education activities. Further, the system 100 allows the industrial professionals to apply with the continuing industrial education activities in order to obtain the accreditation and hence maintain their industrial licenses for practice.

The system 100 includes a hardware processor 604. The system 100 also includes a memory 208 coupled to the hardware processor 604. The memory 208 comprises a set of program instructions in the form of a plurality of subsystems. The plurality of subsystem is configured to be executed by the hardware processor 604.

The plurality of subsystems includes a profile generation subsystem 102. The profile generation subsystem 102 is configured to create a user profile for each of one or more registered users using one or more user credentials. In one embodiment, the one or more registered users belong to any industry. In such embodiment, the one or more registered users may be of two types, an industrial professional, such as a doctor, a nurse, a lawyer, an engineer and the like and an industrial organization such as a hospital, an institute, a nursing school, an engineering college, a law firm and the like. In such embodiment, two separate tabs are provided for logging in as a professional or as an organization.

The one or more user credentials of the one or more registered users' may include and is not restricted to personal details, professional career details, photographs, industrial experience with specialty, credits scores earned and are in need to renew licenses in case license is expired, continuing industrial education activities attended and/or performed by the industrial professional, their active industrial license number; status of their industrial practice such as active or expired license, and profile of the industrial organizations including contact details, continuing industrial education activities developed and delivered by the organization, credits earned by the organization for delivering the continuing industrial education activities, ranking of the organization and the like.

The system 100 provides various features such as view, access or update through such generated user profiles. The profile owner may automatically gain knowledge and awareness about currently occurring continuing industrial education events or activities and apply for attending them, which eventually helps them in obtaining the credit scores.

In one specific embodiment, each of the one or more registered users are assigned an identification label. This identification label is assigned based on information related to each of the one or more registered users, such as, but not limited to, name, contact details or industrial license number which uniquely identifies the one or more registered users. These are the one or more user credentials as provided during profile generation. The identification label may include and not limited to, a bar code, a quick response (QR) code, a numeric code, an alpha-numeric code, a graphical code and the like, which when scanned by an optical device is able to fetch the information related to each of the one or more registered users from a server device and identify each of the one or more registered users. Hence, it becomes digitally easier to identify who has attended or contributed or participated in a particular continuing industrial education activity, by scanning one of the one or more registered user's identification label at the time of registration for the continuing industrial education.

The plurality of subsystems includes an event manager subsystem 104. The event manager subsystem 104 is configured to provide access to continuing industrial education content to the one or more registered users based on the created user profiles. The system 100 continuously fetches matching information related to the continuing industrial education activities, based on each of the one or more registered user's preferences, and transmits such continuing industrial education activities to the corresponding one or more registered users.

In one specific embodiment, an information crawler 218 parses an internet to scan, identify, and collect information related to the continuing industrial education content and associated activities occurring in a specified location. Such information needs to be presented on the system 100 for sharing among specific industrial community. Furthermore, the information crawler 218 continuously gathers information related to the continuing industrial education content and associated activities from the internet. This information is stored in a database 216. Hence, the application 214 also collects the information about the continuing industrial education content and the related activities held by the organizations who are not the users of the application 214, avoiding the chances of missing out any important knowledge source or continuing industrial education activity occurrence.

The continuing industrial education content or related information may include and not limited to, general knowledge about the continuing industrial education, spreading awareness about the continuing industrial education program; activities such as live events, written publications, online programs, audio, video, or other electronic media and the like, industrial organization holding the activities for continuing industrial education with the information related to activities, industrial faculty and organizations developing, reviewing, and delivering contents about the continuing industrial education, and industrial bodies regulating continuing industrial education, and any other necessary information related to the continuing industrial education.

In an embodiment, providing access, to the continuing industrial education content to the one or more registered users, may include a keyword searching module and a filtering module. In an embodiment, the keyword searching module and the filtering module are augmented to generate optimized search recommendations based on at least one of: the created user profile and one or more behavioral patterns of the one or more registered users, using a natural language processing model. The event manager subsystem 104 is further configured to provide AI-based personalized recommendations on the continuing industrial education content in priority using an AI model trained to predict user preferences based on one or more historical data comprising at least one of: one or more industry types, one or more user interests, one or more geographic locations, and one or more educational goals.

The event manager subsystem 104 is also configured to manage one or more tasks associated with the user profiles relating to the continuing industrial education content upon providing the access. The system 100 enables proper control over accessing such stated continuing industrial education content.

In such embodiment, the event manager subsystem 104 manages the one or more tasks for each of the one or more registered users respective to their user profiles. For example, the one or more tasks are related to their continuing industrial education and the associated activities, such as listing the conferences on which each of the one or more registered users is interested in, the time, date and place for each conference, and developing, delivering and organizing events by an associated organization, pushing them notifications or reminders for that event, providing a list of conferences, lectures happening around the industrial professional, or a list of publications to be launched on a particular date, or information such as time, date place of other such activities and informing the one or more registered users via notifications.

Additionally, the system 100 may also help the industrial professionals to directly apply online for the events they are interested to participate and/or perform an activity in the event, such as being a speaker. Directly applying may redirect each of the one or more registered users to a website of the organizations holding the event (whether live or online) or access a particular paper or journal for contribution. Further, the system 100 may provide an online form for the one or more registered users to fill in order to participate in such continuing industrial education activities. The continuing industrial education activities may also include delivering a lecture in an industrial institution, or writing and/or contributing to a journal, participating as a faculty or attending lectures in the industrial institution and the like. Such stated process includes enhancing knowledge of the industrial professional and also helps in maintaining their knowledge, skills, competence and performance. The event manager subsystem 104 is further configured to prompting the one or more registered users to renew an industrial license with the industrial organization based on a time of expiration of the industrial licenses.

At the time of participation in the registered continuing industrial education activity, the user may present his user device 202 in order to digitally scan his/her identification label 406 for marking his/her presence at the activity. Doing this, the presence of the user may be automatically identified during the start, the duration and the end of the activity. Resultantly, the application 214 may help in determining the presence of the user in the activity and give assurance to the organizing medical committee/organization of the user's presence throughout the activity after successful completion of the participation in the activity, the application 214 may also automatically awards the credit scores associated with that particular activity and adds them to the present credit score of the user. Hence, the system 100 automatically accredits the industrial professionals for their participation in the continuing industrial education activities and updates the renewal date of their medical licenses.

The plurality of subsystems also includes a credit generation subsystem 106. The credit generation subsystem 106 is configured to generate a credit score for each of the user profiles based on the one or more tasks completed by the one or more registered users, using an AI model. In one embodiment, each of the one or more registered users is given the credit score based on a number of meetings attended, or conferences participated in, or courses accessed. The credit score is awarded to the participating industrial professionals based on a verification process, either instantly or after some time.

In an embodiment, for generating the credit score for each of the user profile based on the one or more tasks, the credit generation subsystem 106 is configured to analyze the completed one or more tasks using one or more pre-defined weights assigned to each task based on one or more parameters comprising at least one of: complexity of the one or more tasks, relevancy of the one or more tasks to one or more industries, the one or more registered users belong to, time spent on completion of the one or more tasks. In an embodiment, the AI model is learned with the one or more pre-defined weights from one or more historical performance data associated with at least one of: the one or more registered users and one or more industries. In an embodiment, the AI model may include at least one of: logistic regression AI model, linear regression model, decision trees AI model, random forest model, K-nearest neighbor model, and the like.

Exemplary verification criteria for providing the credit score awarded by the organizing industrial bodies or organization may depend upon a subject matter, status of a speaker and quality of papers to be presented in the conference or workshop or seminar or panel discussion. For example, the credit score may be computed as 1 hour theory=1 credit hour and 1-hour hands on practice=2 credit hours and so on. Other exemplary verification criteria for providing the credit score may be such as, for each state or national or international, maximum credit hours is awarded as per the schedule of the conference for participation: (a) Half Day—4 credit hours; (b) One day conference 8 credit hours; (c) Two days conference 16 credit hours; (d) Three days conference 24 credit hours and so on.

Further, examples of verification criteria for providing the credit scores may be any paper published in indexed state or national or international journal entitles an author or co-author for the credit hours. Alternatively, any chapter published in a textbook or update book published by professional bodies entitles the author or co-authors for the credit hours. In another alternate embodiment, speakers or chairman or co-chairman or moderator at any program is given two credit hours per talk in addition to the credit hours allotted for that particular academic activity. An organizer of the program except in-house program is awarded five credit hours and an organizer of the inhouse program is awarded two credit hours. In such embodiment, the paper presentation (oral or poster) in international conferences is awarded six credit hours and in state or national conferences is awarded four credit hours. Alternatively, if the conference is a group work, only first and second investigators shall share the credit hours of six hours; and four hours, if the conference is of state or national conference. Hence, there may be a number of ways for deciding the verification criteria for accreditation by the industrial organization developing and delivering the continuing industrial education.

When all the users, who are the industrial professionals, have successfully completed their participation or contribution in the continuing industrial education activity, the system 100 may also automatically have a list of the industrial professionals who have attended the particular activity and send that list to the particular industrial organization who has held that activity. This can be done by scanning the identification labels of the industrial professionals. Hence, the industrial organization will automatically have a list of attending professionals with their details and industrial license numbers, hence avoiding the hectic use of paperwork. Furthermore, the credit scores are awarded to the participating professionals based on the verification, either instantly or after some time.

The plurality of subsystems also includes a professional development score generation subsystem 108. The professional development score generation subsystem 108 is configured to generate professional development score for each of the user profile based on one or more factors upon completion of the one or more tasks using the AI model. In one embodiment, the one or more factors comprises continuing industrial education history, number of years of schooling and college education, total levels of continuing professional development courses, employment history, types of certifications undertaken and number of associated conferences attended, number of associated white papers published and the like.

The professional development score plays a key role in a talent manager's decision to offer a job and may be used by many organizations to institutions. One metric primarily used in computing a professional development score is knowledge utilization or the percentage of education currently being used in an individual's respective professional experience. In one specific embodiment, a professional development score of 85 or above is generally considered good and may result in a professional receiving a better job profile. Scores greater than 85 are considered excellent. Scores between 70 to 84 are considered to be good. Scores between 69 to 55 are considered to be fair. Scores between 54 to 30 are considered to be fair. Scores between 25 to 10 are considered to be very poor. An individual's professional development score provides statistical analysis of skill worthiness and directly affects professional prospects and pay scale.

Furthermore, the professional development score changes and may rise or fall based on new information. In such embodiment, the professional development score changes due to updating of knowledge on time, by organising knowledge transfer initiatives, by attending more courses and by working with specialized experts to improve professional development score.

The plurality of subsystems also includes a report generation subsystem 110. The report generation system 110 is configured to generate a report on each of the one or more tasks completed by the one or more registered users relating to the continuing industrial education content.

The report generation system 110 is also configured to publish the generated report to the one or more registered users and other industrial authorities via a communication network 222. In such embodiment, each of the one or more registered user may access on the generated report via the user profiles.

The plurality of subsystems further includes a training subsystem 112 configured to train the AI model for providing the AI-based personalized recommendations on the continuing industrial education content in priority. For training the AI model, the training subsystem 112 is configured to obtain one or more training datasets associated with the one or more historical data from the one or more databases 216. The training subsystem 112 is further configured to train the AI model on the one or more training datasets associated with the one or more historical data. The training subsystem 112 is further configured to generate one or more scores for relevancy of each continuing industrial education content based on the trained AI model. The training subsystem 112 is further configured to assign one or more weightages to each continuing industrial education content based on the one or more scores generated for each continuing industrial education content. The training subsystem 112 is further configured to provide the AI-based personalized recommendations on the continuing industrial education content, in priority, to predict the user preferences, based on the one or more weightages assigned to each continuing industrial education content. The training subsystem 112 is further configured to refine the AI model on the prediction of the user preferences through a feedback mechanism based on user activities with the AI-based personalized recommendations on the continuing industrial education content.

In an embodiment, the training subsystem 112 configured to train the AI model for generating the professional development score for each of the user profile. For training the AI model, the training subsystem 112 is configured to obtain one or more second training datasets associated with at least one of: one or more professional development trajectories and one or more accrediting factors, from the one or more databases 216. The training subsystem 112 is further configured to train the AI model on the one or more second training datasets associated with at least one of: the one or more professional development trajectories and the one or more accrediting factors. The training subsystem 112 is further configured to generate one or more scores for the one or more factors comprising at least one of: the continuing industrial education history, the number of years of schooling and college education, the total levels of the continuing professional development courses, and the employment history, based on the trained AI model on the one or more second training datasets.

The training subsystem 112 is further configured to assign one or more second weightages for the one or more factors based on the one or more scores generated for the one or more factors. The training subsystem 112 is further configured to generate the professional development score for each of the user profile based on the one or more second weightages assigned for the one or more factors. The training subsystem 112 is further configured to adapt the AI model to learn and enhance a process of generating the one or more scores for the one or more factors by adding one or more data comprising one or more user feedback and industry-specific standards.

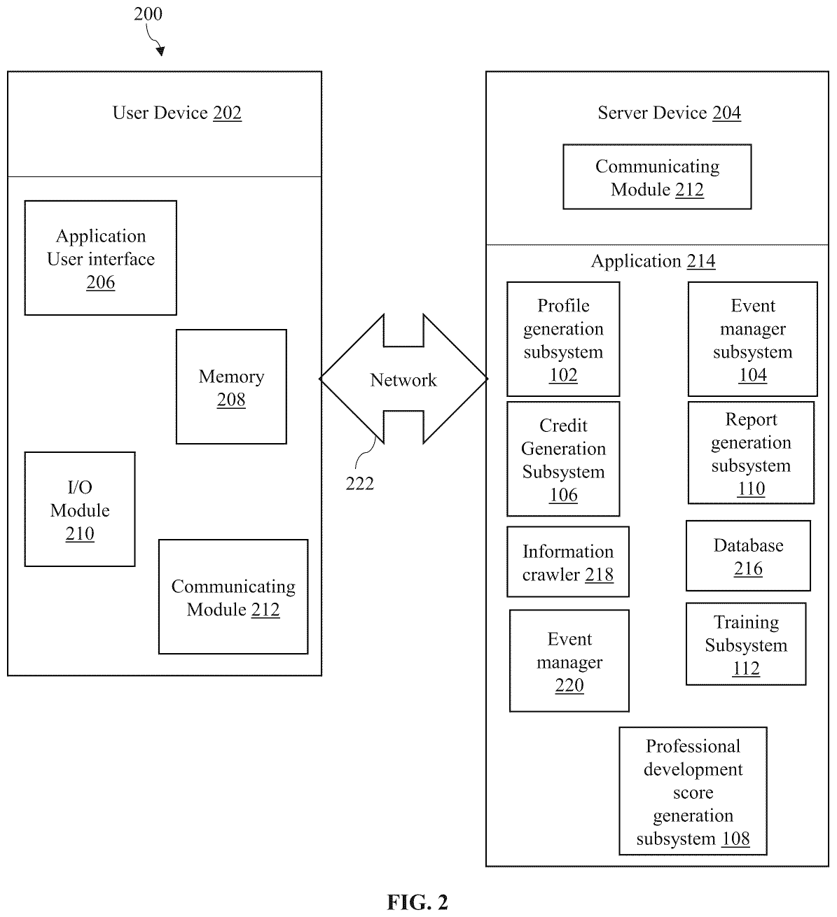

FIG. 2 is a block diagram 200 illustrating an exemplary computing environment comprising the user device 202 and a server device 204 communicating through a communication network 222, in accordance with an embodiment of the present disclosure.

In such embodiment, an industrial professional views and accesses continuing industrial education related information through the user device 202. The user device 202 may include an electronic communication device such as a computer, a laptop, a smart phone, a tablet and the like. In one such embodiment, the user device 202 may run software programs such as an embedded web browser or custom client application, capable of communicating with the server device 204 or input/output module 210. Further, the communication network 222 may include, but is not restricted to, Internet, PSTN, Local Area Network (LAN), Wide Area Network (WAN), and Metropolitan Area Network (MAN).

The computing environment provides the application 214 (also referred to as ‘app’ throughout the detailed description). The application 214 may be a mobile application or a web-based application or both that is run on the server device 204 and also accessible at the user device 202. The application 214 is operated by a user utilizing the user device 202. According to the present disclosure, the application 214 provides an interactive platform which is accessible to the industrial community, including the industrial professionals, such as and not limited to doctors, nurses, pharmacists, engineer, lawyer, or any other industry; and also the medical organizations such as and not limited to hospitals, institutions, nursing schools, other industrial organizations for sharing and accessing information related to the continuing industrial education which is necessary for the industrial professionals to enhance their industrial knowledge throughout their tenure. The continuous industrial education related information may include and not limited to general knowledge about the continuous industrial education spreading awareness about the continuous industrial education program; activities such as live events, written publications, online programs, audio, video, or other electronic media and the like; industrial organization holding the activities for continuous industrial education with the information related to activities; medical faculty and organizations developing, reviewing, and delivering contents about the continuous industrial education; and industrial bodies regulating the continuous industrial education; and any other necessary information related to the continuous industrial education.

The user device 202 includes an application user interface 206, a memory 208, an input/output module 210 (I/O module), and a communicating module 212. The application 214 has the user interface 206 displayed at the user device 202 through which the user may browse for operating the application 214 using the I/O module 210. The I/O module 210 may include and is not restricted to keyboard, touch pad, mouse, camera, speaker, microphone, display screen and the like. The user device 202 has the memory 208 for running and processing the application 214, if the application 214 is a mobile application, and also stores other entities, such as software programs to run the application 214. Further, the user device 202 communicates with the server device 204 using the communicating module 212 via the network 222.

The server device 204 includes the communicating module 212. The application 214 executes on the server device 204. The application 214 consists of the profile generation subsystem 102, the credit generation subsystem 106, the event manager subsystem 104, the professional development score generation subsystem 108, the report generation subsystem 110, the training subsystem 112, the information crawler 218, an event manager 220; and a number of databases 216. The user creates a profile on the application 214 using the profile generation subsystem 102. The credit generation subsystem 106 computes credit scores to be awarded to the user including the industrial professional and/or the organization based on their participation in continuous industrial education activities or organizing such activities. The information crawler 218 parses internet for crawling, scanning, identifying and collecting information related to the continuous industrial education and associated activities occurring in the states, the country or abroad. Such information needs to be presented on the application 108 for sharing among the industrial community.

The event manager 220 manages the one or more tasks for the users respective to their profiles related to their continuous industrial education and the associated activities. The communicating module 212 allows the server device 204 to communicate with the user device 202.

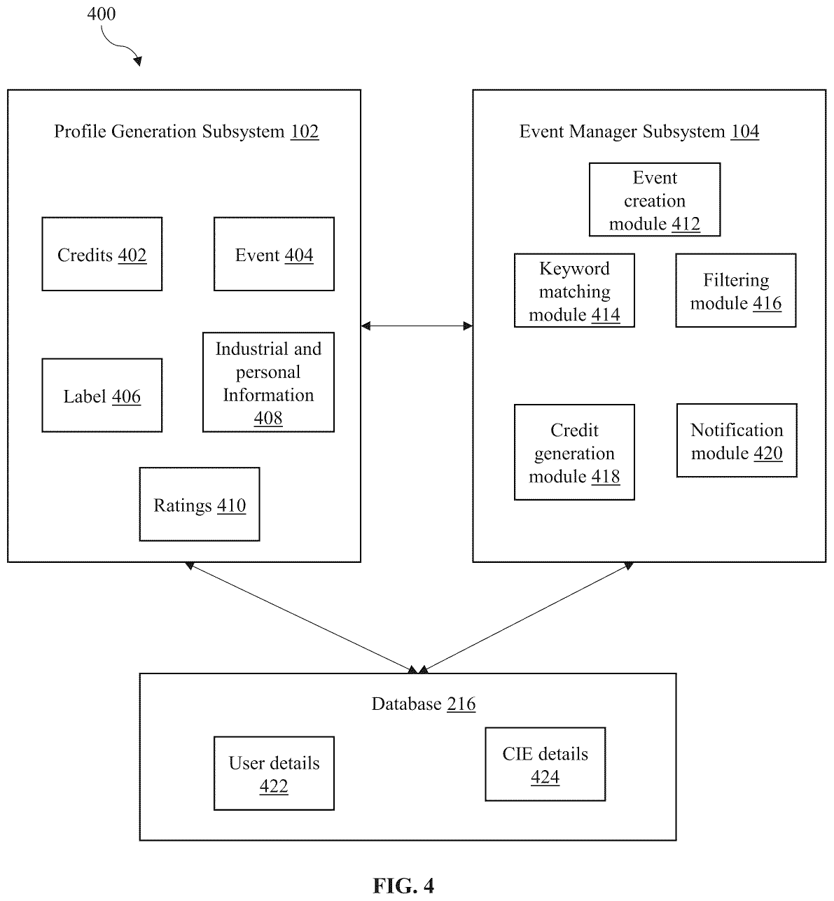

The application 214 comprises the number of databases 216 at the server device 204 that stores all the information regarding the users (professionals and organizations) who have created profiles at the application 214; and also about all the information provided by the regulatory and/or industrial organizations about the continuous industrial education and associated activities; and the information parsed by the information crawler 218 from the internet; and any other necessary information for the knowledge of the continuous industrial education. The number of databases 216 hence stores and maintains information related to the users including the professionals and the organizations and all the necessary information about the continuous industrial education which can inform the users about any activities occurring and educate the users about the necessity of the continuous industrial education. A detailed description about the number of databases 216 is provided in FIG. 4.

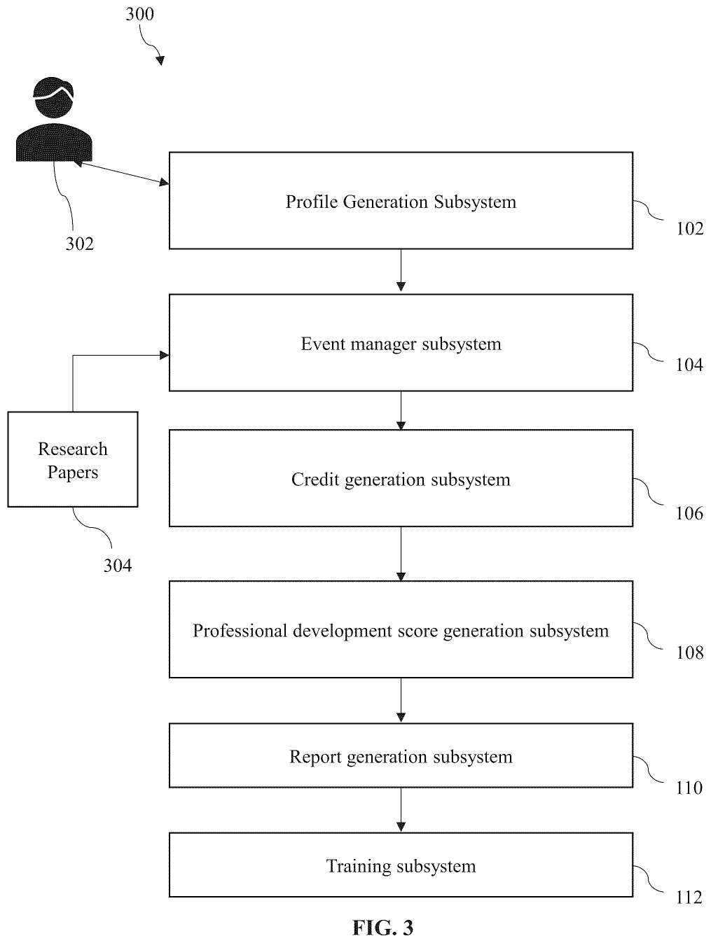

FIG. 3 is a block diagram 300 illustrating another exemplary computing system 100 for accreditation of industrial professionals in accordance with an embodiment of the present disclosure. In the given exemplary embodiment, a medical professional X 302 working in a hospital Y registers in the system 100 to access and learn about new research papers 304 published in a particular health domain. The medical professional X 302 first registers in the system 100 and creates a profile through a profile generation subsystem 102. The medical professional X 302 provides valid credentials during the registration and creation of the user profile.

The medical professional X 302 is provided access to the particular research papers 304 via an event manager subsystem 104. For example, the event manager subsystem 104 may provide access to the research papers 304 on the medical journal based on a particular subject matter. The system 100 via the event manager subsystem 104 further manages one or more tasks of the medical professional X 302 via the generated user profile of the medical professional X 302.

The system 100 via a credit generation subsystem 106 may also generate a credit score for the one or more tasks as executed by the medical professional X 302 while accessing the particular research papers 304. A professional development score is generated by a professional development score generation subsystem 108 for each of the user profile based on one or more factors upon completion of one or more tasks. The credit scores may be used for certification purpose of the medical professional X 302. The system 100 further generates and publishes a report of the one or more tasks via a report generation subsystem 110.

Similarly, the system 100 may behave similarly for any engineering professional or a legal professional, who wishes to get accreditation for professional work. The engineering professional may undergo the industrial education courses as provided by the system 100 and earn requisite credit score for gaining accreditation or certificate for any course or degree.

The profile generation subsystem 102, the event manager subsystem 104, the credit generation subsystem 106, the professional development score generation subsystem 108, the report generation subsystem 110, the training subsystem 112, in FIG. 3 is substantially equivalent to the profile generation subsystem 102, the event manager subsystem 104, the credit generation subsystem 106, the professional development score generation subsystem 108, the report generation subsystem 110, and the training subsystem 112, of FIG. 1.

FIG. 4 illustrates a block diagram 400 of components of the computing system 100 and the interaction among themselves, in accordance with an embodiment of the present invention. The profile generation subsystem 102 is configured to access personal and industrial information 408 from the each of the one or more registered users. During the process of operation of the system 100, the profile generation subsystem 102 helps to manipulate or generate events 404, credit 402, label 406 and ratings 410.

In an embodiment, a user profile briefs an overview of his/her career and personal details. The profile generation subsystem 102 creates a user profile page dedicated to the user that shows user's personal and professional details along with the credit scores awarded to the user. The user profile includes and not limited to the personal and professional details of the user; credit scores earned 402; a list of CME activities/events participated 404; professional status of the user; identification label 406 of the user, and the ratings 410. While creating the profile at the application 214, the application 214 at the first may ask the user whether they are an industrial professional or an organization. This way the application 214 stores the information separately for the industrial professionals and the industrial organizations at the server device 204. Thereafter, the user may fill into the details as asked and aforementioned. An industrial professional has to enter his/her industrial license number, which can indicate his/her industrial status for industrial practice. As the profile is created, the application 214 automatically creates an identification label 406 dedicated to the particular user. The identification label 406 is any mark or representation that contains information related to the user, such as and not limited to name, contact details or industrial license number which uniquely identifies the user. The label 406 may include and not limited to a bar code, a QR code, a numeric code, an alpha-numeric code, a graphical code and the like, which when scanned by an optical device is able to fetch the details from the server device 204 and identify the user. Therefore, any user who creates the profiles at the application 214 can be uniquely identified. Hence, it is digitally easier to identify who has attended or contributed or participated in a particular continuous industrial education activity, by scanning their identification label 406 at the time of registration for the continuous industrial education activity.

Further in an embodiment, the profile generation subsystem 102 may also provide ratings 410, in case of industrial organizations depending on their reputations and contributions to the industrial community and also depending on the number and frequency of the continuous industrial education activities developed and delivered by them.

The application 214 also includes an event manager subsystem 104 which communicates with the profile generation subsystem 102 and manages and communicates necessary tasks or information related to the continuous industrial education content and associated activities to the users. The application 214 continuously stores information related to the users and the continuous industrial education activities in its databases 216. Since, it is obvious that a particular industrial professional with his/her specialty will only be interested to attend or contribute the activity of the continuous industrial education which is either dedicated to his/her specialty or somehow related to the specialty. Therefore, if all the information about every continuous industrial education activity is provided to every user in the application 214 page, then it may or may not be considered as non-useful or spam info. Hence, the event manager subsystem 104 uses an event creation module 412 to create and store a list of related activities helpful for a user to attend.

The event manger subsystem 104 is configured to manage each of the events of the system 100. The keyword matching module 414 and the filtering module 416 are used to fetch a list of matching continuing industrial education activities/events and provide them to each of the one or more registered users. The keyword matching module 414 and the filtering module 416 maps the user's profile which briefs about his/her industrial professional career, and his medical interests provided in the profile, which may or may not be related to his professional domain, with a list of continuous industrial education activities (and/or information) currently happening or about to happen in future, which is available for participation. Further, in an embodiment, the keyword matching module 414 may also present the users with a list of continuous industrial education activities based on their profiles and location and other interests listed in the profile. Hence, the application 214 provides the users with the list of continuous industrial education activities occurring around them. The event manager subsystem 104 sends the list of continuous industrial education activities and information in users' inboxes or messages (not shown in the Figures), from where the users can access the link and apply for registration of the activities.

A notifications module 420 alert the one or more registered users about event details. The notification module 420 may also automatically create a calendar event based on the registration of the user for a particular activity and may also remind the user as the activity time and date approaches. The notifications may be in the form of a message, a call, an email, a calendar reminder or the like.

Furthermore, all the user details 422 and the continuing industrial education (CIE) details 424 are stored in the database 216. The database 216 may be remotely stored or locally stored. The database 216 stores information related to each of the one or more registered users including the industrial professionals and the organizations and all the necessary information about the continuing industrial education which may inform the each of the one or more registered users about any activities occurring and educate each of the one or more registered users about the necessity of the continuing industrial education.

For the whole process to operate, the system 100 is first integrated into various training platforms and then a user logs in and performs training to get accreditation. Initially, an industry specific cabinet first approves or appoints a sole continuous professional development (CPD) reckoner. The appointed continuous professional development (CPD) reckoner directs industry specific councils to integrate various system portal shortcuts in their customized websites for access.

The appointed continuous professional development (CPD) reckoner also creates an advisory board to help the system 100 with achieving set targets. In one embodiment, a group of users acting as an advisory board is created, which further comprises a plurality of subject matter experts. The advisory board provides license renewal support with industry specific cabinet. In one specific embodiment, the plurality of subject matter experts comprises five to seven subject matter experts.

In one embodiment, a plurality of knowledge or technical providers approvals and license renewal support is dynamically linked with industry specific councils. In one such embodiment, at least one member of the advisory board comprises a member from the respective industry specific councils. Moreover, one or more scope of work contracts is generated for each group of users acting as an advisory board member.

The system 100 manages one or more tasks by the plurality of subject matter experts for on boarding of all the plurality of knowledge or technical providers. In such embodiment, a number pre-defined continuous professional development (CPD) programs are registered. In another such embodiment, all knowledge or training providers are made acquainted with sponsorship opportunity and supports. Various sponsorship options as provided by all knowledge or training providers have three months lead time. Furthermore, one or more marketing and public relation activities for the one or more tasks is generated based on the continuous professional development (CPD) programs.

In such exemplary embodiment, the system 100 captures one or more user details for registration of a user and for creation of customized user profile dashboard. The one or more user details comprises at least one of electronic mail, mobile number, practise license, photos and the like. In such embodiment, a payment gateway is dynamically linked for payment from one or more registered user.

In one embodiment, the system 100 enables access of a customized user profile dashboard to one or more registered user after payment. The customized user profile dashboard ensures easy search for continuous professional development (CPD) programs and post continuous professional development (CPD) programs follow ups. In such embodiment, the customized user profile dashboard is navigated with options based on city, zip code, specialty, date, topic and industry affiliation and to choose at least on of continuous professional development (CPD) programs. Against each continuous professional development (CPD) programs credit points along with confirmation possibilities are provided. In such embodiment, the access of the chosen continuous professional development (CPD) programs enables accumulation of credit points.

The knowledge and training programs are listed and when confirmed by the one or more registered users reading materials are provided through email ID. All notifications and updates are sent via the electronic mail. In such embodiment, the industrial activity is attended by the each of the one or more registered users via access code entry. Once assessments are done, the credit scores are provided to each of the one or more registered users.

Additionally, the system 100 provides easy process for license renewal. In one embodiment, each of the one or more registered users is enabled to access and train with the chosen continuous professional development (CPD) programs for a pre-defined period of time to achieve a set value of the credit points. For example, the pre-defined period of time period of time may be five years and the set value of the credit points may be 30 credit points.

In such embodiment, upon achieving the set value of the credit points, each of the one or more registered users is allowed to submit renewal application online form and documents. The documents comprise scanned undertaking certificate, two pass-port size photos, and self-attested permanent registration certificate and the like.

In another embodiment, the training with the chosen continuous professional development (CPD) programs for each of the one or more registered users for the pre-defined period of time is reauthenticated before submission of the renewal application online form and the documents over to the industry specific councils. The industry specific councils approve license after payment of renewal fees and reauthentication. In such embodiment, the system 100 alerts the one or more registered users for download of colour copy of the renewed industrial license upon approval from the industry specific council. Alerts may be provided through electronic mail, mobile messages and the like. The cycle of accumulation of points is repeated for another pre-defined period of time.

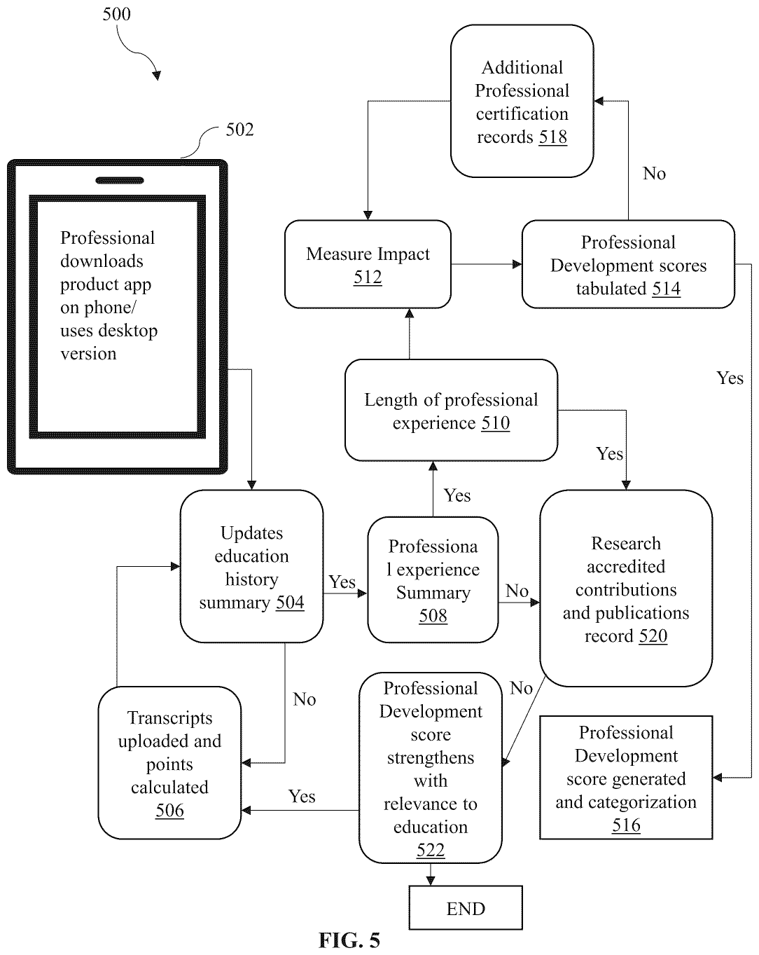

FIG. 5 is a flowchart illustrating an exemplary process 500 of computing the professional development score in accordance with an embodiment of the present disclosure. At step 502, an application of the system 100 is downloaded in a phone or any handheld device by each of the one or more registered user. A desktop version may also be downloaded by Each of the one or more registered user.

At step 504, education history summary of each of the one or more registered user is updated. In such embodiment, at step 506, transcripts are updated, and points are computed if the education history summary of each of the one or more registered user is not updated.

At step 508, professional experience summary is generated for each of the one or more registered user. Further, at step 510, length of professional experience is computed for each of the one or more registered user. In such embodiment, at step 512, an impact is measured from the computed length of professional experience and additional professional certification records. At step 514, the professional development score is tabulated based on the measured impact. At step 516, the professional development score is generated and categorized from the tabulated professional development score. Moreover, at step 518, more additional professional certification records are accepted to re-measure the impact.

At step 520, research accredited contributions and publications records are accepted from each of the one or more registered users to generate professional experience summary. In such embodiment, at step 522, the contribution relevant to education is only used to strengthen the professional development score.

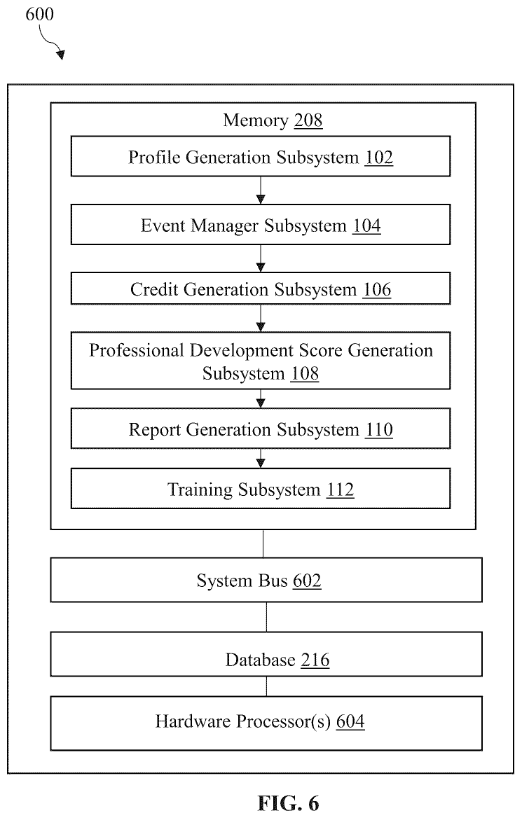

FIG. 6 is a block diagram 600 illustrating components in the computing system 100, such as those shown in FIG. 1, in accordance with an embodiment of the present disclosure. The components comprise the hardware processor 604, the memory 208, the database 216, and a system bus 602.

The hardware processor(s) 604, as used herein, means any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

The memory 208 includes a plurality of subsystems stored in the form of executable program which instructs the hardware processor 604 via the system bus 602 to perform the method steps as illustrated in FIG. 1. The memory 208 has following subsystems: the profile generation subsystem 102, the event manager subsystem 104, the credit generation subsystem 106, professional development score generation subsystem 108, the report generation subsystem 110, and the training subsystem 112. The database 216 is configured to store each of the one or more registered users' information and continuing industrial education content.

The profile generation subsystem 102 is configured to create the user profiles for the one or more registered users using the one or more user credentials. The event manager subsystem 104 is configured to provide the access to continuing industrial education content to the one or more registered users based on the created user profiles. The event manager subsystem 104 is further configured to augment the keyword searching module (i.e., a keyword matching module) 414 and the filtering module 416, to generate the optimized search recommendations based on at least one of: the created user profile and the one or more behavioral patterns of the one or more registered users, using the natural language processing model. The event manager subsystem 104 is further configured to provide the AI-based personalized recommendations on the continuing industrial education content in priority using the AI model trained to predict the user preferences based on the one or more historical data comprising at least one of: the one or more industry types, the one or more user interests, the one or more geographic locations, and the one or more educational goals. The event manager subsystem 104 is also configured is manage the one or more tasks associated with the user profiles relating to the continuing industrial education content upon providing the access.

The credit generation subsystem 106 is configured to generate the credit score for the user profiles based on the one or more tasks completed by the one or more registered users, using the AI model. The professional development score generation subsystem 108 is configured to generate the professional development score for each of the user profile based on the one or more factors upon completion of the one or more tasks using the AI model. The report generation system 110 is configured to generate the report on each of the one or more tasks completed by the one or more registered users relating to the continuing industrial education content. The report generation system 110 is further configured to publish the generated report to each of the one or more registered users and other industrial authorities via the communication network 222.

Computer memory elements may include any suitable memory device(s) for storing data and executable program, such as read only memory, random access memory, erasable programmable read only memory, electrically erasable programmable read only memory, hard drive, removable media drive for handling memory cards and the like. Embodiments of the present subject matter may be implemented in conjunction with program modules, including functions, procedures, data structures, and application programs, for performing tasks, or defining abstract data types or low-level hardware contexts. Executable program stored on any of the above-mentioned storage media may be executable by the hardware processor(s) 604.

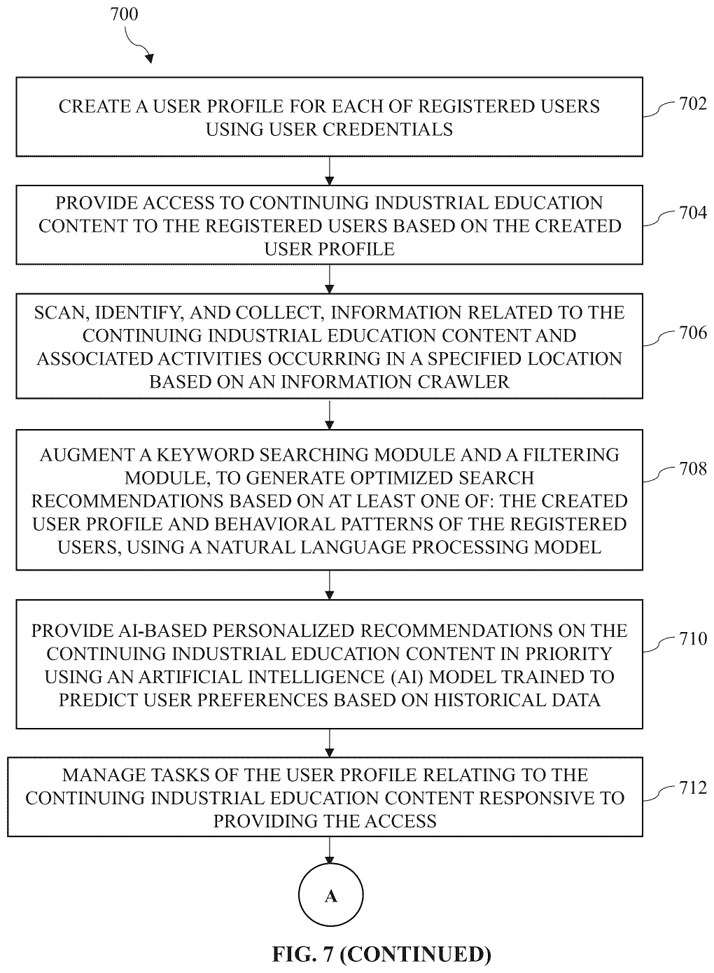

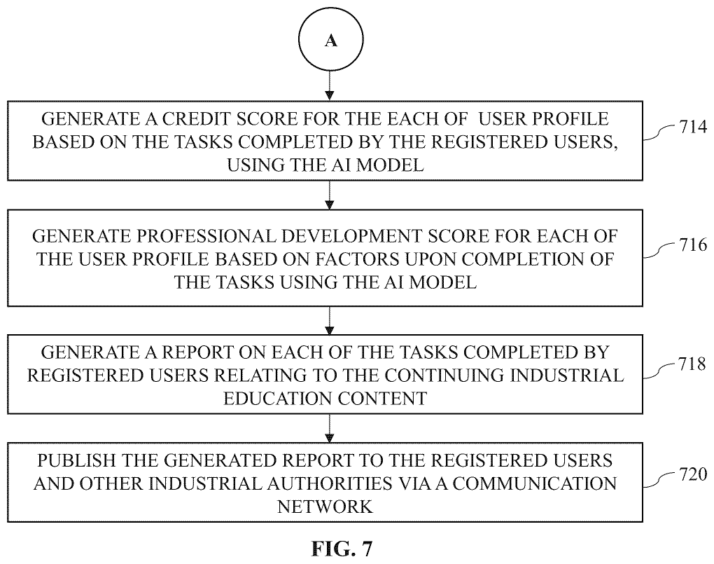

FIG. 7 is a process flowchart illustrating an exemplary AI-based computing method 700 for accreditation of the industrial professionals in accordance with an embodiment of the present disclosure. At step 702, the user profile is created for each of the one or more registered users using the one or more user credentials. In one aspect of the present embodiment, the user profile is created for each of the one or more registered users by the profile generation subsystem 102.

At step 704, the access is provided to the continuing industrial education content to the one or more registered users based on the created user profiles. In one aspect of the present embodiment, the access is provided to the continuing industrial education content by the event manager subsystem 104.

At step 706, the information related to the continuing industrial education content and associated activities occurring in a specified location, are scanned, identified, and collected, based on the information crawler 218. In one embodiment, the one or more tasks of the user profiles comprises listing the conferences on which each of the one or more registered users is interested in, time, date, and place for that conference, and developing, delivering and organizing events by an industrial representative of any associated organization. Furthermore, the process includes storing both information related to each of the one or more registered users and information related to the continuing industrial education content.

At step 708, the keyword searching module or the keyword matching module 414 and the filtering module 416, are augmented to generate the optimized search recommendations based on at least one of: the created user profile and one or more behavioral patterns of the one or more registered users, using the natural language processing model. The process also includes prompting the one or more registered users to renew an industrial license with an industrial organization based on time of expiration of the industrial licenses.

At step 710, the AI-based personalized recommendations on the continuing industrial education content in priority, are provided using the AI model trained to predict the user preferences based on the one or more historical data comprising at least one of: the one or more industry types, the one or more user interests, the one or more geographic locations, and the one or more educational goals.

At step 712, the one or more tasks associated with the user profile relating to the continuing industrial education content are managed upon providing the access. In one aspect of the present embodiment, the one or more tasks associated with the user profile relating to the continuing industrial education content is managed by the event manager subsystem 104.

At step 714, the credit score for each of the user profile are generated based on the one or more tasks completed by the one or more registered users. In one aspect of the present embodiment, the credit score for each of the user profile are generated based on the one or more tasks completed by the one or more registered users by the credit generation subsystem 106.

At step 716, the professional development score is generated for each of the user profile based on the one or more factors upon completion of the one or more tasks. In one aspect of the present embodiment, the professional development score is generated for each of the user profile based on the one or more factors upon completion of the one or more tasks by the professional development score generation subsystem 108. In another aspect of the present embodiment, the one or more factors comprises the continuing industrial education history, the number of years of schooling and college education, the total levels of continuing professional development courses, and the employment history.

At step 718, the report is generated on each of the one or more tasks completed by the one or more registered users relating to the continuing industrial education content. In one aspect of the present embodiment, the report is generated on the one or more tasks completed by the one or more registered users relating to the continuing industrial education content by the report generation subsystem 110.

At step 720, the generated report is published to the one or more registered users and other industrial authorities via the communications network 222. In one aspect of the present embodiment, the generated report is published to the one or more registered users and other industrial authorities by the report generation subsystem 110.

The AI-based computing method 700 for managing one or more tasks associated with the user profile comprises approving and appointing a sole continuous professional development (CPD) reckoner. In such embodiment, the appointed continuous professional development (CPD) reckoner directs industry specific councils to integrate various system portal shortcuts in their customized websites for access. Further, the appointed continuous professional development (CPD) reckoner creates an advisory board to help with achieving set targets. Specific number of knowledge and training programs are planed with the help of subject matter experts, wherein all knowledge and training providers are made acquainted with sponsorship opportunity and supports.

The AI-based computing method 700 for creating the user profile for each of one or more registered users comprises registering for the user profile through an electronic mail ID and a mobile number. In one embodiment, customized dashboard is accessed after payment via a third-party application. In such embodiment, the customized dashboard is navigated with options based on location, date, topic and industry affiliation.

The AI-based computing method 700 for renewal of an industrial license comprises training for certain number of years to fulfil number of requirement years. In such embodiment, after such training necessary documents are submitted for the industrial license renewal to the industry specific council. For renewable of an industrial license, required renewal fees is paid through a payment gateway.

In such embodiment, the continuing industrial education content comprises knowledge regarding the continuing industrial education content, spreading awareness regarding a continuing industrial education program, activities of live events, written publications, online programs, audio, video, and other electronic media and activities comprising developing, reviewing, and delivering contents regarding continuing industrial education.

The present invention provides an interactive platform which is common between the industrial professionals and the industrial organizations and provides them a common provision to share, view and access all the available information related to the continuing industrial education awareness and activities for continuing industrial education. Further, the system 100 thereof provides an automated provision for the industrial professionals to view and be updated with the current knowledge of their industrial fields and also about the continuing industrial education activities. Furthermore, the system provides an automated way of getting updates about the continuing industrial education activities may or may not be based on their preferences, registering for a desired continuing industrial education activity and obtains the credit scores related to that activity. The system 100 provides a professional development score, which may help a professional on career path through their lifetime.

Additionally, the system 100 further provides an automated platform for the industrial bodies to issue notices about the number of continuing industrial education activities they are organizing and call for participants either by announcing the notifications on the interface of the system 100, or by pushing notifications to the preferred professionals. Additionally, the system 100 provides the organizing industrial bodies an automated and digitized way of accreditation which avoids the needs of hectic paperwork and organizing things on paper. Thereby, the system 100 provides industrial education compliance platform along with centralized tracking and support system. The system 100 may easily track fees and attendance of any registered user.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer-readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the system 100 either directly or through intervening I/O controllers. Network adapters may also be coupled to the system 100 to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/system 100 in accordance with the embodiments herein. The system 100 herein comprises at least one processor or central processing unit (CPU). The CPUs are interconnected via the system bus 602 to various devices including at least one of: a random-access memory (RAM), read-only memory (ROM), and an input/output (I/O) adapter. The I/O adapter can connect to peripheral devices, including at least one of: disk units and tape drives, or other program storage devices that are readable by the system 100. The system 100 can read the inventive instructions on the program storage devices and follow these instructions to execute the methodology of the embodiments herein.

The system 100 further includes a user interface adapter that connects a keyboard, mouse, speaker, microphone, and/or other user interface devices including a touch screen device (not shown) to the bus to gather user input. Additionally, a communication adapter connects the bus to a data processing network, and a display adapter connects the bus to a display device which may be embodied as an output device including at least one of: a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that are issued on an application based here on. Accordingly, the embodiments of the present invention are intended to be illustrative, but not limiting, of the scope of the invention, which is set forth in the following claims.

What is claimed is:

1. An artificial intelligence based (AI-based) computing system for accreditation of industrial professionals, the AI-based computing system comprising:
a hardware processor; and
a memory coupled to the hardware processor, wherein the memory comprises a set of program instructions in a form of a plurality of subsystems, configured to be executed by the hardware processor, wherein the plurality of subsystems comprises:
a profile generation subsystem configured to create a user profile for each of one or more registered users using one or more user credentials, wherein the one or more registered users belong to one or more industries,
wherein the profile generation subsystem is further configured to assign an identification label to each of the created user profile, wherein the identification label comprises contact details of the one or more registered users and an industrial license number, and wherein the identification label is associated with at least one of: a bar code, a quick response (QR) code, a numeric code, an alpha-numeric code, and a graphical code;
an event manager subsystem configured to:
provide access to continuing industrial education content to the one or more registered users based on the created user profile, wherein the AI-based computing system comprises an information crawler configured to scan, identify, and collect information related to the continuing industrial education content and associated activities occurring in a specified location,
wherein providing access comprises a keyword searching module and a filtering module, wherein the keyword searching module and the filtering module are augmented to generate optimized search recommendations based on at least one of: the created user profile and one or more behavioral patterns of the one or more registered users, using a natural language processing model; and
provide AI-based personalized recommendations on the continuing industrial education content in priority using an AI model trained to predict user preferences based on one or more historical data comprising at least one of: one or more industry types, one or more user interests, one or more geographic locations, and one or more educational goals;
manage one or more tasks associated with the user profile relating to the continuing industrial education content responsive to providing the access, wherein managing the one or more tasks comprises a continuous development reckoner, and wherein the continuous development reckoner achieves set targets, provides license renewal support, and plans specific number of knowledge and training programs;
a credit generation subsystem configured to generate a credit score for each of the user profile based on the one or more tasks completed by the one or more registered users, using the AI model;
a professional development score generation subsystem configured to generate professional development score for each of the user profile based on one or more factors upon completion of the one or more tasks using the AI model, wherein the one or more factors comprises at least one of: continuing industrial education history, number of years of schooling and college education, total levels of continuing professional development courses, and employment history; and
a report generation subsystem configured to:
generate one or more reports on each of the one or more tasks completed by the one or more registered users relating to the continuing industrial education content; and
publish the generated one or more reports to the one or more registered users and other industrial authorities via a communications network.

2. The AI-based computing system of claim 1, further comprising a training subsystem configured to train the AI model for providing the AI-based personalized recommendations on the continuing industrial education content in priority, wherein in training the AI model, the training subsystem is configured to:
obtain one or more training datasets associated with the one or more historical data from one or more databases;
train the AI model on the one or more training datasets associated with the one or more historical data;
generate one or more scores for relevancy of each continuing industrial education content based on the trained AI model;
assign one or more weightages to each continuing industrial education content based on the one or more scores generated for each continuing industrial education content;
provide the AI-based personalized recommendations on the continuing industrial education content, in priority, to predict the user preferences, based on the one or more weightages assigned to each continuing industrial education content; and
refine the AI model on the prediction of the user preferences through a feedback mechanism based on user activities with the AI-based personalized recommendations on the continuing industrial education content.

3. The AI-based computing system of claim 1, wherein the one or more tasks of the user profiles comprises listing conferences on which each of the one or more registered users is interested in, a time, a date and a place for each conference, and developing, delivering and organizing events by an industrial representative of an associated organization.

4. The AI-based computing system of claim 1, wherein the credit score is generated based on a number of meetings, conferences and courses accessed by each of the one or more registered users.

5. The AI-based computing system of claim 1, wherein in generating the credit score for each of the user profile based on the one or more tasks, the credit generation subsystem is configured to analyze the completed one or more tasks using one or more pre-defined weights assigned to each task based on one or more parameters comprising at least one of: complexity of the one or more tasks, relevancy of the one or more tasks to one or more industries, the one or more registered users belong to, time spent on completion of the one or more tasks, and
wherein the AI model is learned with the one or more pre-defined weights from one or more historical performance data associated with at least one of: the one or more registered users and one or more industries.

6. The AI-based computing system of claim 1, further comprising a database configured to store information related to each of the one or more registered users and information related to the continuing industrial education content.

7. The AI-based computing system of claim 1, wherein the continuing industrial education content comprises continuing medical education.

8. The AI-based computing system of claim 1, wherein the training subsystem is further configured to train the AI model for generating the professional development score for each of the user profile, wherein in training the AI model, the training subsystem is configured to:
obtain one or more second training datasets associated with at least one of: one or more professional development trajectories and one or more accrediting factors, from the one or more databases;
train the AI model on the one or more second training datasets associated with at least one of: the one or more professional development trajectories and the one or more accrediting factors;
generate one or more scores for the one or more factors comprising at least one of: the continuing industrial education history, the number of years of schooling and college education, the total levels of the continuing professional development courses, and the employment history, based on the trained AI model on the one or more second training datasets;
assign one or more second weightages for the one or more factors based on the one or more scores generated for the one or more factors;
generate the professional development score for each of the user profile based on the one or more second weightages assigned for the one or more factors; and
adapt the AI model to learn and enhance a process of generating the one or more scores for the one or more factors by adding one or more data comprising one or more user feedback and industry-specific standards.

9. The AI-based computing system of claim 1, wherein the continuing industrial education content comprises knowledge regarding continuing industrial education content, spreading awareness regarding a continuing industrial education program, activities of live events, written publications, online programs, audio, video, and other electronic media and activities comprising developing, reviewing, and delivering content regarding continuing industrial education.

10. The AI-based computing system of claim 1, wherein the event manager subsystem is further configured for prompting the one or more registered users to renew an industrial license with an industrial organization based on a time of expiration of the industrial licenses.

11. An artificial intelligence based (AI-based) computing method for accreditation of industrial professionals, the AI-based computing method comprising:
creating, by one or more hardware processors, a user profile for each of one or more registered users using one or more user credentials, wherein the one or more registered users belong to one or more industries;
assigning, by the one or more hardware processors, an identification label to each of the created user profile, wherein the identification label comprises contact details of the one or more registered users and an industrial license number, and wherein the identification label is associated with at least one of: a bar code, a quick response (QR) code, a numeric code, an alpha-numeric code, and a graphical code;
providing, by the one or more hardware processors, access to continuing industrial education content to the one or more registered users based on the created user profile;
scanning, identifying and collecting, by the one or more hardware processors, information related to the continuing industrial education content and associated activities occurring in a specified location based on an information crawler;
augmenting, by the one or more hardware processors, a keyword searching module and a filtering module, to generate optimized search recommendations based on at least one of: the created user profile and one or more behavioral patterns of the one or more registered users, using a natural language processing model;
providing, by the one or more hardware processors, AI-based personalized recommendations on the continuing industrial education content in priority using an AI model trained to predict user preferences based on one or more historical data comprising at least one of: one or more industry types, one or more user interests, one or more geographic locations, and one or more educational goals;
managing, by the one or more hardware processors, one or more tasks associated with the user profile relating to the continuing industrial education content responsive to providing the access, wherein managing the one or more tasks comprises a continuous development reckoner, and wherein the continuous development reckoner achieves set targets, provides license renewal support, and plans specific number of knowledge and training programs;
generating, by the one or more hardware processors, a credit score for each of the user profile based on the one or more tasks completed by the one or more registered users, using the AI model;
generating, by the one or more hardware processors, professional development score for each of the user profile based on one or more factors upon completion of the one or more tasks using the AI model, wherein the one or more factors comprises at least one of: continuing industrial education history, number of years of schooling and college education, total levels of continuing professional development courses, and employment history;
generating, by the one or more hardware processors, one or more reports on each of the one or more tasks completed by the one or more registered users relating to the continuing industrial education content; and
publishing, by the one or more hardware processors, the generated one or more reports to the one or more registered users and other industrial authorities via a communications network.

12. The AI-based computing method of claim 11, further comprising training, by the one or more hardware processors, the AI model for providing the AI-based personalized recommendations on the continuing industrial education content in priority, wherein training the AI model comprises:
obtaining, by the one or more hardware processors, one or more training datasets associated with the one or more historical data from one or more databases;
training, by the one or more hardware processors, the AI model on the one or more training datasets associated with the one or more historical data;
generating, by the one or more hardware processors, one or more scores for relevancy of each continuing industrial education content based on the trained AI model;
assigning, by the one or more hardware processors, one or more weightages to each continuing industrial education content based on the one or more scores generated for each continuing industrial education content;
providing, by the one or more hardware processors, the AI-based personalized recommendations on the continuing industrial education content, in priority, to predict the user preferences, based on the one or more weightages assigned to each continuing industrial education content; and
refining by the one or more hardware processors, the AI model on the prediction of the user preferences through a feedback mechanism based on user activities with the AI-based personalized recommendations on the continuing industrial education content,
wherein the continuing industrial education content comprises knowledge regarding the continuing industrial education content, spreading awareness regarding a continuing industrial education program, activities of live events, written publications, online programs, audio, video, and other electronic media and activities comprising developing, reviewing, and delivering contents regarding continuing industrial education, and
wherein the continuing industrial education content comprises continuing medical education.

13. The AI-based computing method of claim 11, wherein the credit score is generated based on a number of meetings, conferences and courses accessed by each of the one or more registered users.

14. The AI-based computing method of claim 11, wherein generating the credit score for each of the user profile based on the one or more tasks, comprises analyzing, by the one or more hardware processors, the completed one or more tasks using one or more pre-defined weights assigned to each task based on one or more parameters comprising at least one of: complexity of the one or more tasks, relevancy of the one or more tasks to one or more industries, the one or more registered users belong to, time spent on completion of the one or more tasks,
wherein the AI model is learned with the one or more pre-defined weights from one or more historical performance data associated with at least one of: the one or more registered users and one or more industries, and
wherein the one or more tasks of the user profiles comprises listing the conferences on which each of the one or more registered users is interested in, a time, a date, and a place for each conference, and developing, delivering and organizing events by an industrial representative of associated organization.

15. The AI-based computing method of claim 11, further comprising training, by the one or more hardware processors, the AI model for generating the professional development score for each of the user profile, wherein training the AI model, comprises:
obtaining, by the one or more hardware processors, one or more second training datasets associated with at least one of: one or more professional development trajectories and one or more accrediting factors, from the one or more databases;
training, by the one or more hardware processors, the AI model on the one or more second training datasets associated with at least one of: the one or more professional development trajectories and the one or more accrediting factors;
generating, by the one or more hardware processors, one or more scores for the one or more factors comprising at least one of: the continuing industrial education history, the number of years of schooling and college education, the total levels of the continuing professional development courses, and the employment history, based on the trained AI model on the one or more second training datasets;
assigning, by the one or more hardware processors, one or more second weightages for the one or more factors based on the one or more scores generated for the one or more factors;
generating, by the one or more hardware processors, the professional development score for each of the user profile based on the one or more second weightages assigned for the one or more factors; and
adapting, by the one or more hardware processors, the AI model to learn and enhance a process of generating the one or more scores for the one or more factors by adding one or more data comprising one or more user feedback and industry-specific standards.

16. The AI-based computing method of claim 11, further comprising prompting the one or more registered users to renew an industrial license with an industrial organization based on a time of expiration of the industrial licenses.

17. The AI-based computing method of claim 11, wherein for managing the one or more tasks associated with the user profile comprises:
creating, by the one or more hardware processors, a group of users acting as an advisory board comprising a plurality of subject matter experts, wherein at least one member of the advisory board comprises a member from respective industry specific councils;
dynamically linking, by the one or more hardware processors, a plurality of knowledge and technical providers approvals and license renewal support with industry specific councils, generating one or more scope of work contracts for each group of users acting as an advisory board member;
managing, by the one or more hardware processors, the one or more tasks by the plurality of subject matter experts for on boarding of the plurality of knowledge and technical providers;
registering, by the one or more hardware processors, for a number of pre-defined continuous professional development (CPD) programs; and
generating, by the one or more hardware processors, one or more marketing and public relation activities for the one or more tasks based on the continuous professional development (CPD) programs.

18. The AI-based computing method of claim 11, wherein creating the user profile for each of the one or more registered users comprises:
capturing, by the one or more hardware processors, one or more user details for registration of a user and for creation of customized user profile dashboard, wherein the one or more user details comprises at least one of electronic mail, mobile number, practise license and photos;
dynamically linking, by the one or more hardware processors, a payment gateway for payment from the one or more registered user;
enabling, by the one or more hardware processors, an access of a customized user profile dashboard to one or more registered user after payment, wherein the customized user profile dashboard ensures easy search for continuous professional development (CPD) programs and post continuous professional development (CPD) programs follow ups;
navigating, by the one or more hardware processors, the customized user profile dashboard with options based on city, zip code, specialty, date, topic and industry affiliation to select at least one of continuous professional development (CPD) programs; and
enabling, by the one or more hardware processors, an access of the selected continuous professional development (CPD) programs for accumulation of credit points.

19. The AI-based computing method of claim 18, further comprises renewing an industrial license for the one or more registered users by:
enabling, by the one or more hardware processors, an access to each of the one or more registered users and train the one or more registered users with the selected continuous professional development (CPD) programs for a pre-defined period of time to achieve a set value of the credit points;
allowing, by the one or more hardware processors, each of the one or more registered users to submit renewal application online form and documents upon achieving the set value of the credit points, wherein the documents comprise at least one of scanned undertaking certificate, two pass-port size photos, and self-attested permanent registration certificate;
reauthenticating, by the one or more hardware processors, the training with the chosen continuous professional development (CPD) programs for the pre-defined period of time before submission of the renewal application online form and the documents over to the industry specific councils, wherein the industry specific councils approve license after payment of renewal fees and reauthentication; and
alerting, by the one or more hardware processors, the one or more registered users for downloading a colour copy of renewed industrial license upon approval from the industry specific council.

20. A non-transitory computer-readable storage medium having instructions stored therein that, when executed by a hardware processor, cause the hardware processor to perform method steps comprising:
creating a user profile for each of one or more registered users using one or more user credentials, wherein the one or more registered users belong to one or more industries,
assigning an identification label to each of the created user profile, wherein the identification label comprises contact details of the one or more registered users and an industrial license number, and wherein the identification label is associated with at least one of: a bar code, a quick response (QR) code, a numeric code, an alpha-numeric code, and a graphical code;
providing access to continuing industrial education content to the one or more registered users based on the created user profile;
scanning, identifying and collecting, information related to the continuing industrial education content and associated activities occurring in a specified location based on an information crawler;
augmenting a keyword searching module and a filtering module, to generate optimized search recommendations based on at least one of: the created user profile and one or more behavioral patterns of the one or more registered users, using a natural language processing model;
providing AI-based personalized recommendations on the continuing industrial education content in priority using an AI model trained to predict user preferences based on one or more historical data comprising at least one of: one or more industry types, one or more user interests, one or more geographic locations, and one or more educational goals;
managing one or more tasks associated with the user profile relating to the continuing industrial education content responsive to providing the access, wherein managing the one or more tasks comprises a continuous development reckoner, and wherein the continuous development reckoner achieves set targets, provides license renewal support, and plans specific number of knowledge and training programs;
generating a credit score for each of the user profile based on the one or more tasks completed by the one or more registered users, using the AI model;
generating professional development score for each of the user profile based on one or more factors upon completion of the one or more tasks using the AI model, wherein the one or more factors comprises at least one of: continuing industrial education history, number of years of schooling and college education, total levels of continuing professional development courses, and employment history;
generating one or more reports on each of the one or more tasks completed by the one or more registered users relating to the continuing industrial education content; and
publishing the generated one or more reports to the one or more registered users and other industrial authorities via a communications network.
